(19) United States
(12) Patent Application Publication
(10) Pub. No.: US 2024/0376087 A1
(43) Pub. Date: Nov. 14, 2024

(54) PYRAZOLE AMIDE BASED COMPOUNDS AND USES AGAINST BREAST CANCER THEREOF

(71) Applicant: COUNCIL OF SCIENTIFIC AND INDUSTRIAL RESEARCH AN INDIAN REGISTERED BODY INCORPORATED UNDER THE REGN., Deihi New Deihi (IN)

(72) Inventors: Priya SULOCHANA, Kerala Thiruvananthapuram (IN); Somappa SASIDHAR BALAPPA, Kerala Thiruvananthapuram (IN); Raghu KOZHIPARAMBIL GOPALAN, Kerala Thiruvananthapuram (IN); Lakshmi SREERENJINI, Kerala Thiruvananthapuram (IN); Ashitha KIZHAKKAN THIRUTHI, Kerala Thiruvananthapuram (IN)

(21) Appl. No.: 18/686,652

(22) PCT Filed: Nov. 16, 2022
(86) PCT No.: PCT/IN2022/051006

§ 371 Date: Feb. 26, 2024

(30) Foreign Application Priority Data
Nov. 16, 2021 (IN) 202111052657

(51) Int. Cl. C07D 409/12; A61K 31/4155; A61P 35/00; C07D 231/14

(52) U.S. Cl. CPC C07D 409/12; A61K 31/4155; A61P 35/00; C07D 231/14

(57) ABSTRACT

The present disclosure provides new derivatives of pyrazole amide represented by the formula (Formula I) in which when R=—CH₂—, then R₁ may be one among benzene, 4-methyl benzene, 4-methoxy benzene, 4-chloro benzene, 4-fluoro benzene, 2-chloro benzene, 3,4-dichloro benzene, 3,5-trifluoromethylbenzene, and when R=—SO₂—, then R may be one among benzene, 4-methoxy benzene, 4-chloro benzene, 4-bromo benzene, 4-trifluoromethyl benzene, 3-trifluoromethyl benzene, 2-bromo benzene, 2-chlorothiophen, 2,3-dichlorothiophen substituents. The disclosure also relates to compounds of formula (I) for its use as an anticancer agent in triple negative breast cancer (TNBC) cells. The disclosure established the effectiveness of the pyrazole amide derivative of formula I (I-8), against TNBC cells by inducing cell death by various mechanisms through interaction with diverse cellular pathways.

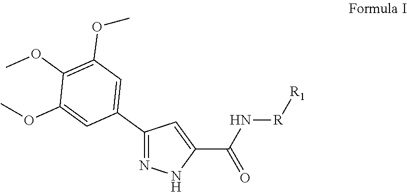

### FIELD OF THE INVENTION

The present invention relates to compound of formula I which are useful as anticancer agent in triple negative breast cancer (TNBC) cells.

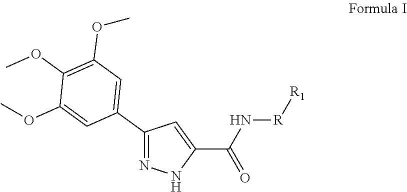

Particularly, the present invention relates to a pyrazole amide based small compound of formula I which functions as anticancer agent in triple negative breast cancer (TNBC) cells. More particularly, the present invention established the effectiveness of one of the pyrazole amide compounds of formula I (I-8), against TNBC cells by inducing cell death by various mechanisms through interaction with diverse cellular pathways.

### BACKGROUND AND PRIOR ART OF THE INVENTION

The burden of cancer incidence ranks as a leading cause of mortality worldwide. According to the recent reports by GLOBOCAN 2020, an estimate of 19.3 million new cancer cases and 10 million cancer deaths are reported worldwide in 2020. Female breast cancer has surpassed lung cancer as the most commonly diagnosed cancer. 2.3 million new cases of breast cancer are reported in 2020 (11.7% of total diagnosed) and the reported death rate was 6.9%. It is the fifth leading cause of cancer mortality worldwide with 685000 deaths. As per the population-based cancer registries and hospital-based cancer registries across India, the projected number of cancer patients in India in 2020 is 1392179 with common sites as breast, lung, mouth, cervix and tongue. The death rates for female breast and cervical cancers were significantly higher in developing countries compared to developed countries. The global burden of cancer is expecting a 47% rise in 2040 when compared to 2020 with a larger increase in developing countries (64-95%) when compared to developed countries (32-56%). Therefore, necessary efforts should build in developing countries in near future for the dissemination of preventive measures and giving proper cancer care which utmost needed for global cancer control (Sung et al., Global Cancer Statistics 2020: GLOBOCAN Estimates of incidence and mortality worldwide for 36 Cancers in 185 countries. CA Cancer J Clin. 2021, 71 (3): 209-249. doi: 10.3322/caac.21660, Mathur et al., Cancer Statistics, 2020: Report from National Cancer Registry Programme, India. JCO Glob Oncol. 2020 6:1063-1075. doi: 10.1200/GO.20.00122).

Breast cancer is the second leading cause of cancer death in women. It is broadly divided into three major subtypes based on the presence of absence of molecular markers for estrogen or progesterone receptors, human epidermal growth factor 2 (HER2) and triple negative, which lacks the three standard molecular markers. The advancements in diagnosis and treatment options have increased the survival rate of breast cancer patients dramatically. Adjuvant, neoadjuvant and targeted chemotherapy (CDK4/6 inhibitor, kinase inhibitor, mTOR inhibitor, PI3K inhibitor etc.), immunotherapy (PD-1 inhibitor, PDL-1 inhibitor) are usually employed for treating breast cancer. Doxorubicin, epirubicin, paclitaxel, docetaxel, 5-FU, cyclophosphamide and carboplatin are generally used chemo drugs for early-stage adjuvant or neoadjuvant therapy. Paclitaxel, docetaxel, abraxane, cisplatin, doxorubicin, eribulin, ixabepilone, vinorelbine etc. are used for advanced breast cancer treatments. Drug combinations are often used to treat early-stage breast cancer whereas single chemo drugs are more used for advanced breast cancer (Information retrieved through cancer.net site). Collaboration among public and private entities and the education on the importance of early diagnosis for breast cancer is prerequisite for successful breast cancer treatment (Al-Mahmood et al., Metastatic and triple-negative breast cancer: challenges and treatment options. Drug Deliv Transl Res. 2018, 8 (5): 1483-1507. doi: 10.1007/s13346-018-0551-3, Mutebi et al., Breast cancer treatment: A phased approach to implementation. Cancer. 2020 May 15; 126 Suppl 10:2365-2378. doi: 10.1002/cncr.32910).

Triple negative breast cancer (TNBC) accounts for 15-20% of total case diagnosed, lack all the hormone receptors. So, hormone therapy and drugs that target HER2 are not useful and chemotherapy is the main systemic treatment option. Metastatic triple negative is the most aggressive form and there is an interest in finding new medications to treat this. Patients with TNBC have a higher rate of distant recurrence and poor prognosis and almost all patients die of even with adjuvant chemotherapy. Various subtypes are identified within TNBC itself with the help of omics-based methods and expression analysis of limited set of genes from TNBC patient samples (WO 2019/112966). This makes the TNBC treatment even complicated. Number of genetic predictive biomarkers are discovered in TNBC which are of diagnostic and prognostic in nature (WO 2016/037009 A1). A variety of chemodrugs are used for TNBC treatment; doxorubicin with cyclophosphamide, doxorubicin with cyclophosphamide and 5FU, paclitaxel and docetaxel. Anthracycline-taxane chemotherapy is the first line treatment whereas carboplatin is considered for BRACA positive TNBC (Pandy et al., Triple negative breast cancer and platinum-based systemic treatment: a meta-analysis and systematic review. BMC Cancer 19, 1065 (2019). https://doi.org/10.1186/s12885-019-6253-5). Identification of immune related targets in TNBC allowed to develop promising immunotherapeutic strategies using inhibitors of PD-1 and PDL-1 (Oualla et al., Immunotherapeutic Approaches in Triple-Negative Breast Cancer: State of the Art and Future Perspectives. Int J Breast Cancer. 2020 Nov. 4:2020:8209173. doi: 10.1155/2020/8209173). In March 2019, FDA have given accelerated approval for the immunotherapy drug atezolizumab (PDL1 inhibitor) in combination with chemotherapy drug abraxane for metastatic triple negative breast cancer. Addition of the PD-1 monoclonal antibody pemrolizumab to platinum containing neoadjuvant chemotherapy resulted in a significant increase in the patient response (Schmid et al., Atezolizumab plus nab-paclitaxel as first-line treatment for unresectable, locally advanced or metastatic triple-negative breast cancer (IMpassion130): updated efficacy results from a randomised, double-blind, placebo-controlled, phase 3 trial. Lancet Oncol. 2020, 21 (1): 44-59. doi: 10.1016/S1470-2045 (19) 30689-8). In April 2020, FDA have given approval for Sacituzumab govitecan (an antibody coupled to the chemo drug irinotecan) for some patients with TNBC. Administration of CDK19 inhibitors in TNBC patients observed a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, or progression free survival (WO 2019/055977 A1). Kidney associated antigen 1 (KAAG1) is an antigen overexpressed in TNBC, and a high affinity antibody can be used for targeting these cells (WO 2013/104050 A3).

Thienotriazolodiazepine based compounds are acting against TNBC in presence of other mitotic or mTOR inhibitors (WO 2015/169951 A1). Pharmacophore which are multikinase inhibitors are acting against TNBC (WO 2015/181201 A1). The important vitamin D analogue AMPI-109 have remarkable cancer specific characteristics which inhibit TNBC cell proliferation and induce apoptosis (US 2015/0202173 A1).

Chemotherapy using platinum-based drugs are effective in TNBC treatment, however more than 40 specific side effects can limit its use. The long term of chemo drugs has severe side effects and often result in multidrug resistance. Also, so far, we do not have a specific cancer drug, each drug is more or less toxic to normal cells as well.

Thus, there is a need for identifying new drugs which are more selective to cancer cells and the identification of their molecular mechanism become crucial.

### OBJECTIVES OF THE INVENTION

Main object of the present invention is to provide a pyrazole amide compound of formula I.

Another object of the present invention is to provide a process for the preparation of pyrazole amide compound of formula I.

Yet another object of the present invention is to evaluate the cytotoxicity of pyrazole amide compound of formula I in TNBC cells.

Yet another object of the present invention is to evaluate the apoptosis induction of the potential pyrazole amide derivatives of formula I in MDA MB 231 cells and the mechanism involved.

Yet another object of the present invention is to evaluate the detachment induced cell death (anoikis) in MDA MB 231 cells by the potential pyrazole amide derivatives of formula I.

Yet another object of the present invention is to evaluate the histone deacetylase 1 (HDAC-1) inhibitory potential of the potential pyrazole amide derivatives of formula I in MDA MB 231 cells.

Yet another object of the present invention is to evaluate the cell cycle inhibitory power of the potential pyrazole amide derivatives of formula I in MDA MB 231 cells.

Yet another object of the present invention is to evaluate the antimetastatic effects in MDA MB 231 cells by the potential pyrazole amide derivatives of formula I.

Yet another object of the present invention is to evaluate the effects on metabolic inhibition exerted by the potential pyrazole amide derivatives of formula I in MDA MB 231 cells.

Yet another object of the present invention is to evaluate the interactive ability with EGF receptor in order to exert the effects by the potential pyrazole amide derivatives of formula I.

Yet another object of the present invention is to study the major pathways affected by the potential pyrazole amide derivatives of formula I, through the transcriptome analysis.

Yet another object of the present invention is to evaluate the combination of the potential pyrazole amide derivatives of formula I with paclitaxel to enhance the overall effectiveness.

### SUMMARY OF THE INVENTION

Accordingly, present invention provides a pyrazole amide-based compound of formula I

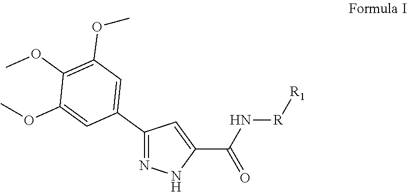

wherein,
when R=—CH₂, then R₁ is selected from the group consisting of benzene, 4-methyl benzene, 4-methoxy benzene, 4-chloro benzene, 4-fluoro benzene, 2-chloro benzene, 3,4-dichloro benzene, 3,5-trifluoromethyl benzene; and
when R=—SO₂, then R₁ is selected from the group consisting of benzene, 4-methoxy benzene, 4-chloro benzene, 4-bromo benzene, 4-trifluoromethyl benzene, 3-trifluoromethyl benzene, 2-bromo benzene, 2-chlorothiophen or 2,3-dichlorothiophen.

In an embodiment of the present invention, said compound is useful as anticancer agent in triple negative breast cancer (TNBC) cells.

In another embodiment of the present invention, the compound of formula I is selected from the group consisting of:

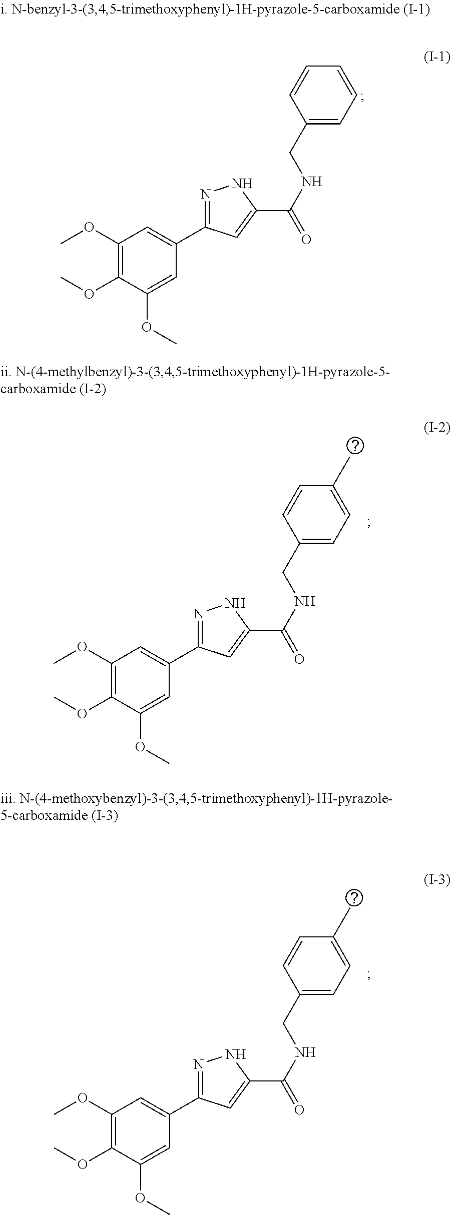

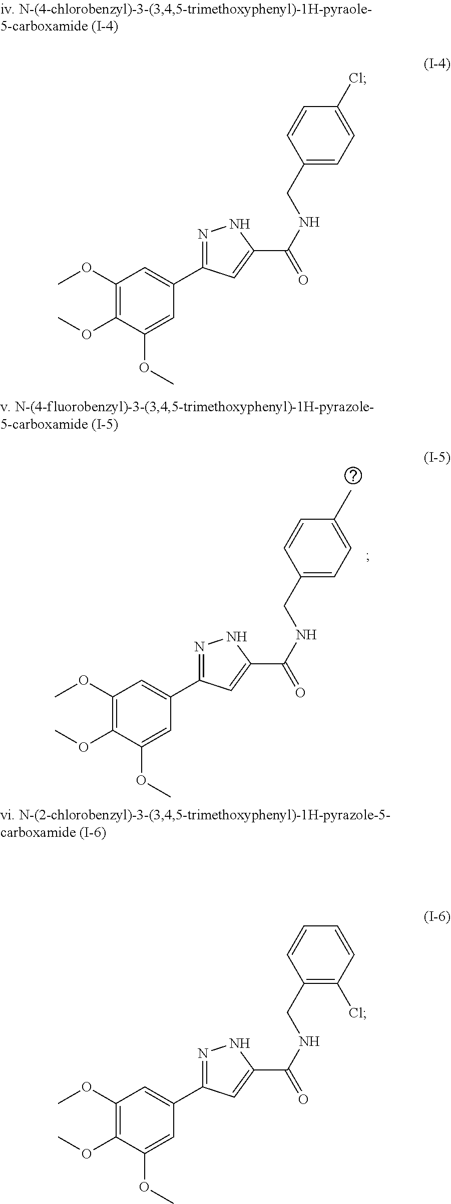

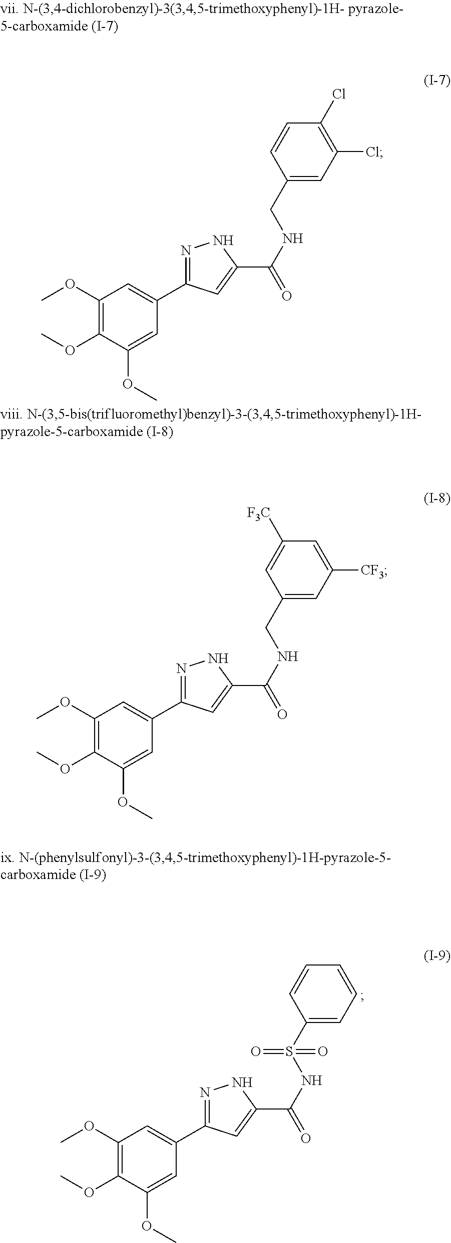 [SMILES: COc1cc(-c2cc(C(=O)NCc3ccc(Cl)c(Cl)c3)[nH]n2)cc(OC)c1OC ; COc1cc(-c2cc(C(=O)NCc3cc(C(F)(F)F)cc(C(F)(F)F)c3)[nH]n2)cc(OC)c1OC ; COc1cc(-c2cc(C(=O)NS(=O)(=O)c3ccccc3)[nH]n2)cc(OC)c1OC]

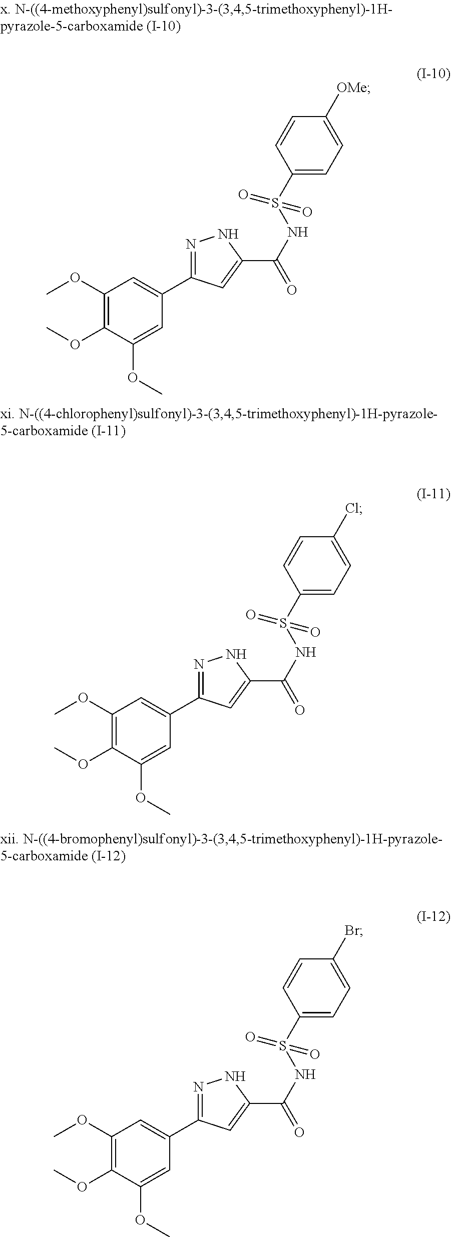 [SMILES: COc1ccc(S(=O)(=O)NC(=O)c2cc(-c3cc(OC)c(OC)c(OC)c3)n[nH]2)cc1 ; COc1cc(-c2cc(C(=O)NS(=O)(=O)c3ccc(Cl)cc3)[nH]n2)cc(OC)c1OC ; COc1cc(-c2cc(C(=O)NS(=O)(=O)c3ccc(Br)cc3)[nH]n2)cc(OC)c1OC]

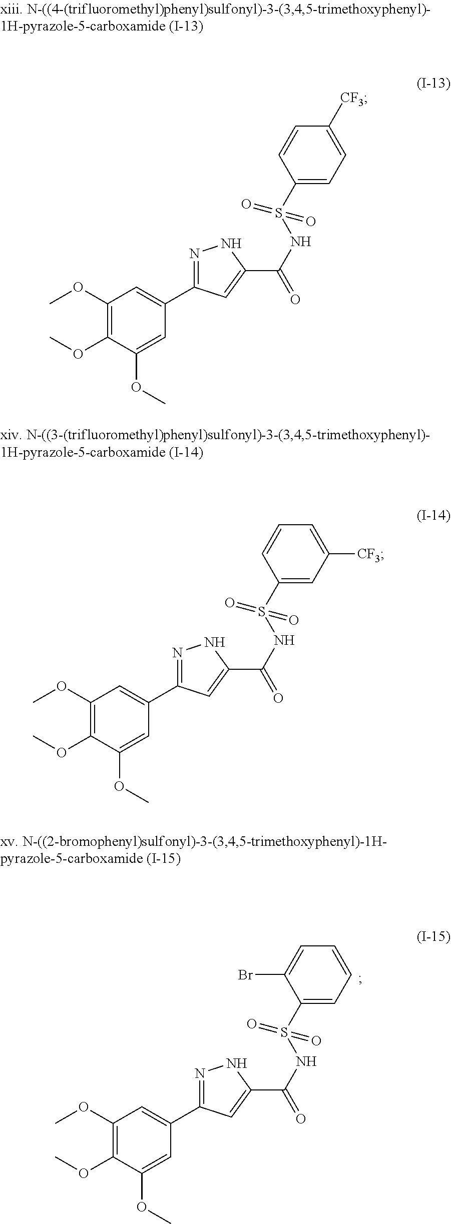 [SMILES: COc1cc(-c2cc(C(=O)NS(=O)(=O)c3ccc(C(F)(F)F)cc3)[nH]n2)cc(OC)c1OC ; COc1cc(-c2cc(C(=O)NS(=O)(=O)c3cccc(C(F)(F)F)c3)[nH]n2)cc(OC)c1OC ; COc1cc(-c2cc(C(=O)NS(=O)(=O)c3ccccc3Br)[nH]n2)cc(OC)c1OC]

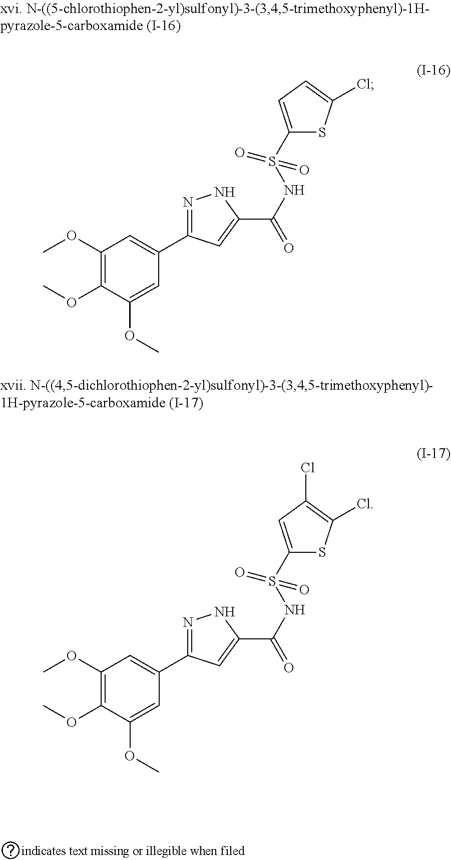 [SMILES: COc1cc(-c2cc(C(=O)NS(=O)(=O)c3ccc(Cl)s3)[nH]n2)cc(OC)c1OC ; COc1cc(-c2cc(C(=O)NS(=O)(=O)c3cc(Cl)c(Cl)s3)[nH]n2)cc(OC)c1OC]

In another embodiment, present invention provides a process for the preparation of compound of formula I comprising the steps of:
i. condensing compound of formula 1 with diethyl oxalate to get ethyl 2,4-dioxo-4-(3,4,5-trimethoxyphenyl) butanoate of formula 2;

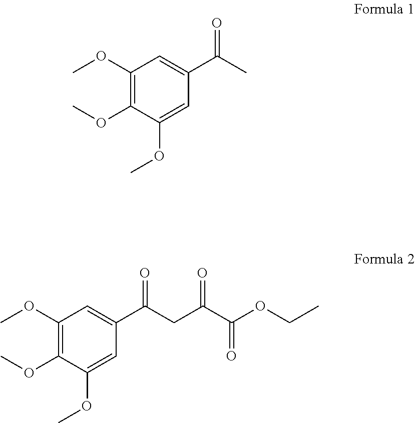 [SMILES: COc1cc(C(C)=O)cc(OC)c1OC ; CCOC(=O)C(=O)CC(=O)c1cc(OC)c(OC)c(OC)c1]

ii. cyclocondensating compound 2 as obtained in step (i) with hydrazine hydrate hydrochloride to form pyrazole ester compound of formula 3;

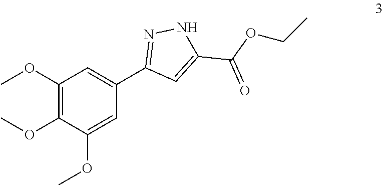 [SMILES: CCOC(=O)c1cc(-c2cc(OC)c(OC)c(OC)c2)n[nH]1]

iii. base mediated hydrolysis of compound of formula 3 as obtained in step (ii) to obtain pyrazole acid compound of formula 4 followed by amide coupling with amine of formula 5 [R1-R—NH₂] to obtain compound of formula I.

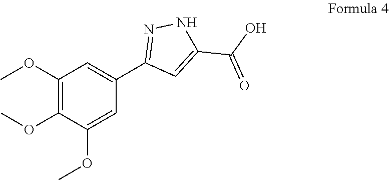 [SMILES: COc1cc(-c2cc(C(=O)O)[nH]n2)cc(OC)c1OC]

In yet another embodiment of the present invention, the compound I-8 induces cytotoxicity in TNBC cells (MDA MB 231) with a 50% growth inhibition value, GI₅₀ of 15.08 μM without having cytotoxicity in normal fibroblasts.

In yet another embodiment of the present invention, the compound I-8 induces apoptosis like morphology changes in MDA MB 231 cells as evidenced by phase contrast and electron microscopy images.

In yet another embodiment of the present invention, said compound induces apoptosis in TNBC cells MDA MB 231 cells (by the activation of both intrinsic and extrinsic pathway).

In yet another embodiment of the present invention, the compound I-8 induces cell cycle arrests at S phase in TNBC cells MDA MB 231 cells.

In yet another embodiment of the present invention, the compound I-8 induces detachment induced cell death (anoikis) in TNBC cells MDA MB 231 cells.

In yet another embodiment of the present invention, the compound I-8 have substantial histone deacetylase 1 (HDAC-1) inhibitory activity phase in TNBC cells (MDA MB 231).

In yet another embodiment of the present invention, the compound I-8 have antimetastatic affects in TNBC cells MDA MB 231 cells.

In yet another embodiment of the present invention, the compound I-8 have metabolic inhibition in TNBC cells MDA MB 231 cells.

In yet another embodiment of the present invention, the compound I-8 interact with EGF receptor in order to exert the effects in TNBC cells (MDA MB 231).

In yet another embodiment of the present invention, the compound I-8 affects the signaling pathways responsible for ER stress induced apoptosis, anoikis and cell cycle arrest as revealed by transcriptome analysis.

In yet another embodiment of the present invention, the compound I-8 results in the clustering of upregulated and downregulated genes into 6 major clusters each for the regulation of stress induced apoptosis, UPR response, autophagy, integrin mediated signalling and cell cycle regulation.

In yet another embodiment of the present invention, the compound I-8 enhances the effectiveness of paclitaxel, 10 times lower concentration of paclitaxel required.

In yet another embodiment of the present invention, the compound I-8 induced nuclear fragmentation and phosphatidyl serine translocation in TNBC cells (MDA MB 231).

In yet another embodiment of the present invention, the compound I-8 induced caspase 3 and caspase 9 activities in TNBC cells (MDA MB 231).

In yet another embodiment of the present invention, the compound I-8 induced reactive oxygen species generation and loss of mitochondrial membrane potential in TNBC cells (MDA MB 231).

In yet another embodiment of the present invention, the compound I-8 upregulated 1337 genes and downregulated 1642 genes as revealed by transcriptome analysis in TNBC cells (MDA MB 231).

In yet another embodiment of the present invention, the compound I-8 upregulated the genes involved in unfolded protein response, p53 transcriptional gene networks, oxidative stress, autophagy, IRE-1 alpha activated chaperones, HATS acetylates histones, programmed cell death and ATR activate gene response in response to ER stress in TNBC cells (MDA MB 231).

In yet another embodiment of the present invention, the compound I-8 downregulated the genes involved in inflammation pathway, glycolysis/gluconeogenesis, ATR pathway, Wnt signaling pathway, Hedgehog pathway, G1-S specific transcription, FAK mediated PI3K/AKT/mTOR pathway, ECM-receptor interaction, TGF beta signaling, Aurora B pathway, PLK-1 pathway, cholesterol biosynthetic pathway, signaling by GPCR, oxidative phosphorylation, Focal adhesion, ALK-1 pathway, α5β3 and α4β1 integrin pathways, extracellular matrix organization, angiogenesis and syndecan 1 pathway in TNBC cells (MDA MB 231).

In yet another embodiment of the present invention, the compound I-8 upregulated genes were organized as 6 protein clusters to perform the functions in TNBC cells (MDA MB 231).

In yet another embodiment of the present invention, the compound I-8 downregulated genes were organized as another 6 protein clusters to perform the functions in TNBC cells (MDA MB 231).

In yet another embodiment of the present disclosure, the compound I-8 enhances the effectiveness of the standard drug paclitaxel (10 times lower concentration of paclitaxel required in presence of the compound I-8).

### BRIEF DESCRIPTION OF THE FIGURES

FIG. 1 represents the schematic diagram for the synthesis of pyrazole amide derivatives of formula I, in accordance with an implementation of the present invention.

FIG. 2A represents phase contrast microscopic images of MDA MB 231 cells treated with different concentrations of I-8 and the comparison with the standard drug paclitaxel (50 nM), in accordance with an implementation of the present invention.

FIG. 2B represents scanning electron microscopy (SEM) images of MDA MB 231 cells treated with I-8 (25 μM) and paclitaxel (50 nM), in accordance with an implementation of the present invention.

FIG. 3 represents phase contrast images of normal fibroblast cells (WI30) cells treated with different concentrations of I-8 and the comparison with the standard drug paclitaxel (50 nM), in accordance with an implementation of the present invention.

FIG. 4A represents fluorescent images showing the nuclear fragmentation of MDA MB 231 cells treated with different concentrations of I-8 and paclitaxel (50 nM), in accordance with an implementation of the present invention.

FIG. 4B represents fluorescent images showing the phosphatidyl serine translocation in MDA MB 231 cells treated with different concentrations of I-8 and paclitaxel (50 nM), in accordance with an implementation of the present invention.

FIG. 5 represents graphical representations of the activation of caspase 3 and caspase 9 in MDA MB 231 cells treated with different concentrations of I-8 and paclitaxel (50 nM), in accordance with an implementation of the present invention.

FIG. 6A represents FACS analysis showing the upregulation of reactive oxygen species (ROS) production in MDA MB 231 cells treated with different concentrations of I-8 and paclitaxel (50 nM), in accordance with an implementation of the present invention.

FIG. 6B represents fluorescent images showing the loss of mitochondrial membrane potential in MDA MB 231 cells treated with different concentrations of I-8 and paclitaxel (50 nM), in accordance with an implementation of the present invention.

FIG. 7 represents protein array showing the upregulation of pro-apoptotic proteins and down-regulation of antiapoptotic process on treatment with I-8, in accordance with an implementation of the present invention.

FIG. 8 represents Anoikis inducing effects of I-8 at different concentrations and paclitaxel (50 nM), in accordance with an implementation of the present invention.

FIG. 9 represents FACS images showing the cell cycle arrest at S phase and the western blot analysis showing the downregulation of cell cycle regulatory proteins involved on treatment with I-8, in accordance with an implementation of the present invention.

FIG. 10 represents graphical representation showing the HDAC inhibitory effects of I-8, in accordance with an implementation of the present invention.

FIG. 11 represents antimetastatic effects of I-8 as revealed by inhibition of cell migration, colony formation and MMP-9 production, in accordance with an implementation of the present invention.

FIG. 12 represents FACS images showing the downregulation of glucose uptake upon treatment with different concentrations of I-8 and paclitaxel (50 nM), in accordance with an implementation of the present invention.

FIG. 13 represents fluorescent images showing the downregulation of EGFR expression upon treatment with different concentrations of I-8 and paclitaxel (50 nM), in accordance with an implementation of the present invention.

FIG. 14 represents volcano plot and clustered heat map showing the differential expression of genes upon treatment with I-8, in accordance with an implementation of the present invention.

FIG. 15 represents gene set enrichment analysis (upregulated genes in transcriptome analysis) for the upregulated pathways upon treatment with I-8, in accordance with an implementation of the present invention.

FIG. 16 represents gene set enrichment analysis (downregulated genes in transcriptome analysis) for the downregulated pathways upon treatment with I-8, in accordance with an implementation of the present invention.

FIG. 17 represents protein cluster and function analysis of upregulated genes upon treatment with I-8, in accordance with an implementation of the present invention.

FIG. 18 represents protein cluster and function analysis of downregulated genes upon treatment with I-8, in accordance with an implementation of the present invention.

FIG. 19 represents phase contrast images showing the chemo sensitization potential of I-8 in combination with paclitaxel, in accordance with an implementation of the present invention.

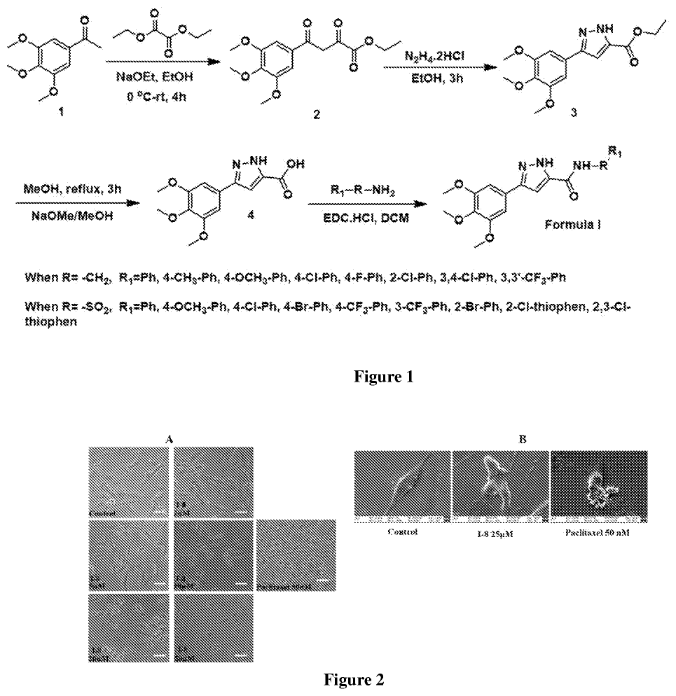

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally equivalent products, compositions, and methods are clearly within the scope of the disclosure, as described herein.

As discussed in the background of the present disclosure, chemotherapy using platinum-based drugs are effective in TNBC treatment, however more than 40 specific side effects can limit its use. The long-term use of chemo drugs possesses severe side effects and often result in multidrug resistance. Additionally, so far, a specific cancer drug has not been discovered, each drug is more or less toxic to normal cells as well. Thus, there is an unmet need for identifying new drugs which are more selective to cancer cells and the identification of their molecular mechanism become crucial.

In an embodiment of the present invention there is provided a pyrazole amide compound of formula I

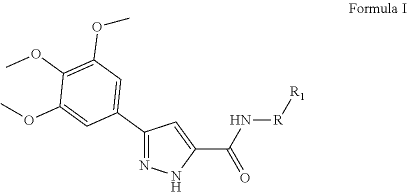

wherein, when R=—CH₂, then R₁ is selected from the group consisting of benzene, 4-methyl benzene, 4-methoxy benzene, 4-chloro benzene, 4-fluoro benzene, 2-chloro benzene, 3,4-dichloro benzene, and 3,5-trifluoromethyl benzene; and when R=—SO₂, then R₁ is selected from the group consisting of benzene, 4-methoxy benzene, 4-chloro benzene, 4-bromo benzene, 4-trifluoromethyl benzene, 3-trifluoromethyl benzene, 2-bromo benzene, 2-chlorothiophen or 2,3-dichlorothiophen, which are more specific to triple negative breast cancer cells and with chemo sensitization potential.

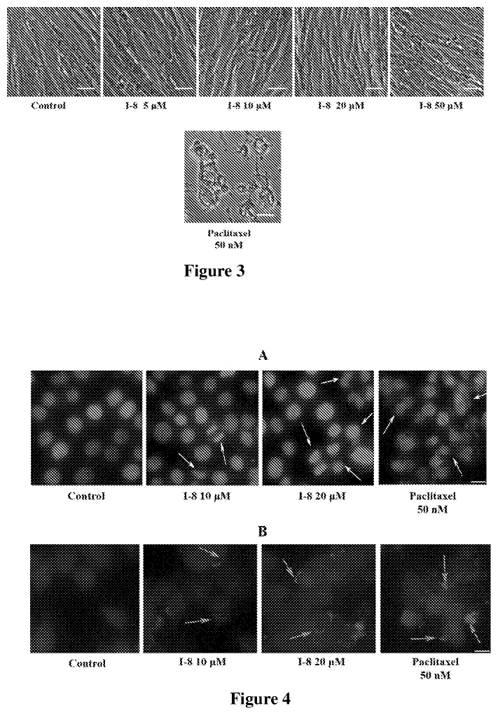

Compounds of formula I, I-1 to I-17 were evaluated for the anticancer potential in triple negative breast cancer cells (MDA MB 231). The preliminary cytotoxicity studies were conducted by MTT assay for all the synthesized compounds (I-1 to 1-17). The GI₅₀ value of the compound I-8 [N-(3,5-bis(trifluoromethyl)benzyl)-3-(3,4,5-trimethoxyphenyl)-1H-pyrazole-5-carboxamide] was found to be 15.08 μM. The compound exerted significant morphological changes in the cells similar to that of apoptosis which was studied by phase contrast and electron microscopy images (FIG. 2A and FIG. 2B).

The effects of I-8 on the toxicity of normal fibroblast cells were assessed and found that that no toxic (FIG. 3).

I-8 showed significant nuclear fragmentation and phosphatidyl serine translocation. Nuclear fragmentation and phosphatidyl serine translocation are key features of apoptosis which were studied by DAPI staining and annexin V staining. (FIG. 4A and FIG. 4B).

I-8 upregulated caspase activity. The cells undergoing apoptosis release the enzymes caspases for the process execution. Caspase 3 and caspase 9 activities were measured in I-8 treated cells and the results were significant (FIG. 5).

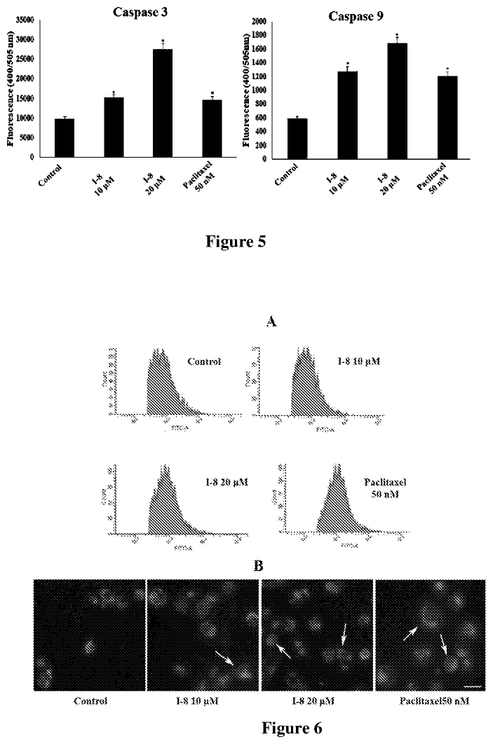

I-8 upregulated ROS generation and loss of mitochondrial membrane potential. The intrinsic pathway of apoptosis involve mitochondria and loss of mitochondrial membrane potential and excess ROS generation are key features of this. In I-8 treated cells, there observed significant generation of ROS and loss of mitochondrial membrane potential (FIG. 6A and FIG. 6B).

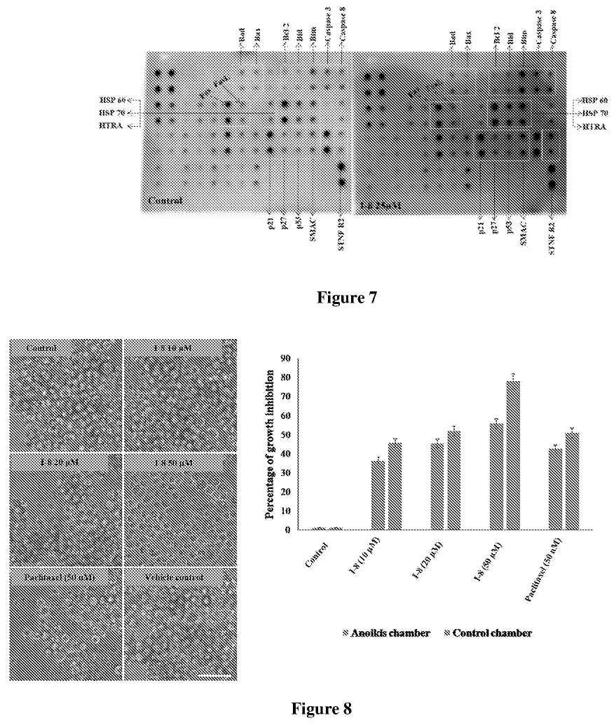

I-8 upregulated the proteins involved in intrinsic and extrinsic pathways. Protein array experiments by western blotting showed the upregulation of key proteins involved in apoptosis regulation. The upregulated proteins in the extrinsic and intrinsic pathways are Bad, Bax, Bid, Bim, caspase3, caspase8, cytochrome c, Fas, Fas ligand, HSP60, HSP70, HTRA, p21, p27, p53, SMAC, STNFR1 and XIAP (FIG. 7).

I-8 induced detachment induced cell death (anoikis). The percentage of growth inhibition in the control chamber and anoikis chamber decreased significantly upon treatment with I-8 (FIG. 8).

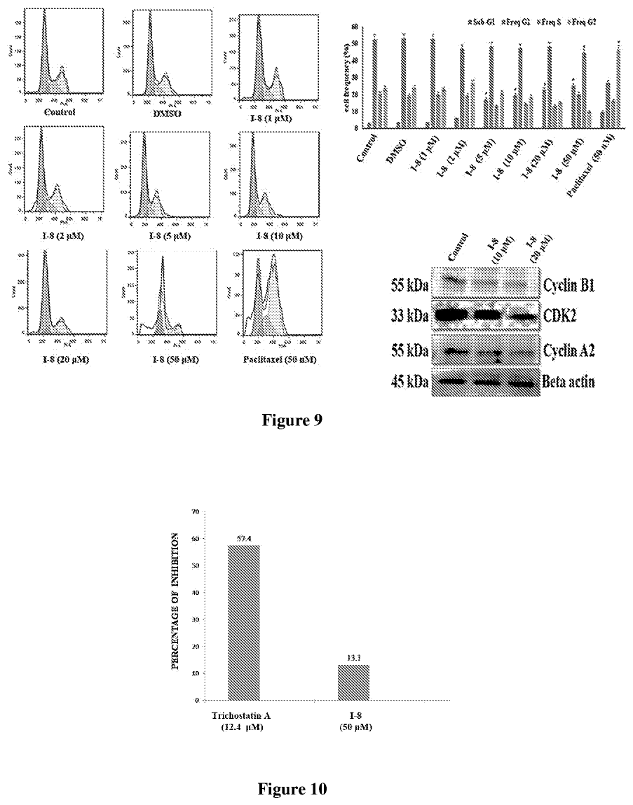

I-8 induced cell cycle arrest at S phase. The distribution of cells in the different phases were monitored by FACS after labelling with PI. The cell cycle regulatory proteins of G1-S transition such as cyclin B1, cyclin A2 and CDK were downregulated in western blot (FIG. 9).

I-8 showed HDAC inhibition. The HDAC inhibitory activity measured by the enzyme assay showed significant inhibition (FIG. 10).

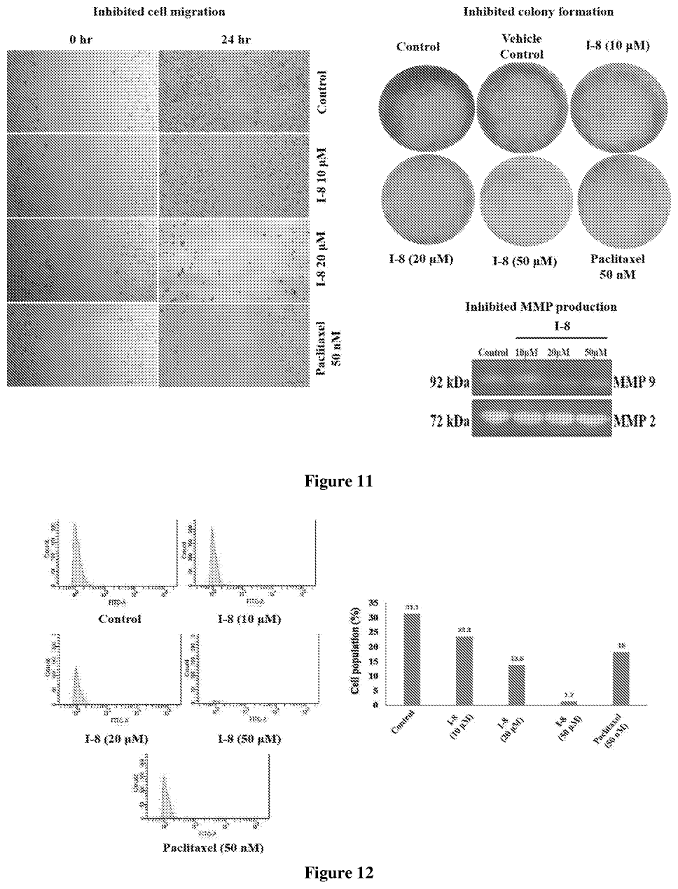

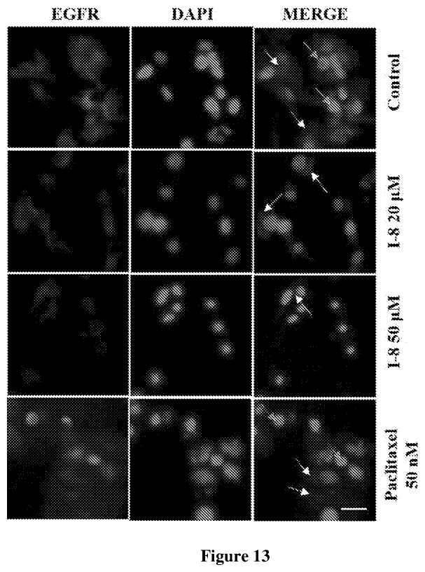

I-8 showed antimetastatic effects. The cell migration inhibition was evident from scratch wound assay experiment, inhibition of colony formation and MMP-9 activity were also reduced (FIG. 11).

I-8 induced metabolic inhibition. The glucose uptake studies by FACS analysis showed significant reduction in the uptake of glucose (FIG. 12).

I-8 showed reduction in EGFR expression. The fluorescent images showed a reduction in the EGFR label (FIG. 13).

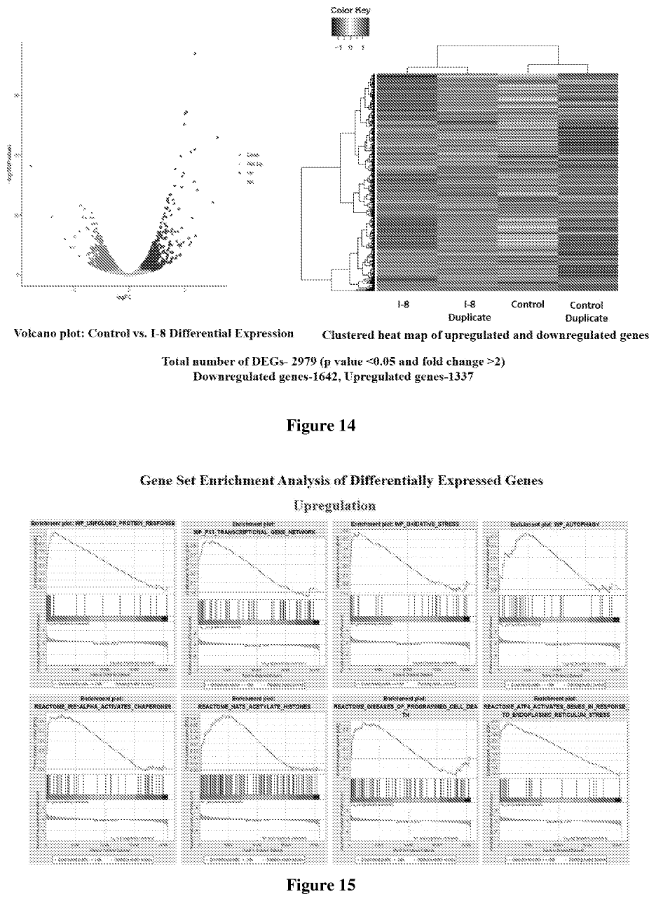

I-8 showed the upregulation of 1337 genes and downregulation of 1642 genes in the whole transcriptome analysis. The isolation of RNA from the control and I-8 treated cells, their transcriptome sequencing using illumina technology resulted in the extraction of differentially expressed genes (FIG. 14).

I-8 treatment showed the upregulation of signaling pathways of ER stress induced apoptosis, UPR response, autophagy, HDAC acetylation etc. Gene Set Enrichment analysis was conducted by comparing the upregulated genes with canonical set of target genes and identified the upregulated pathways (FIG. 15).

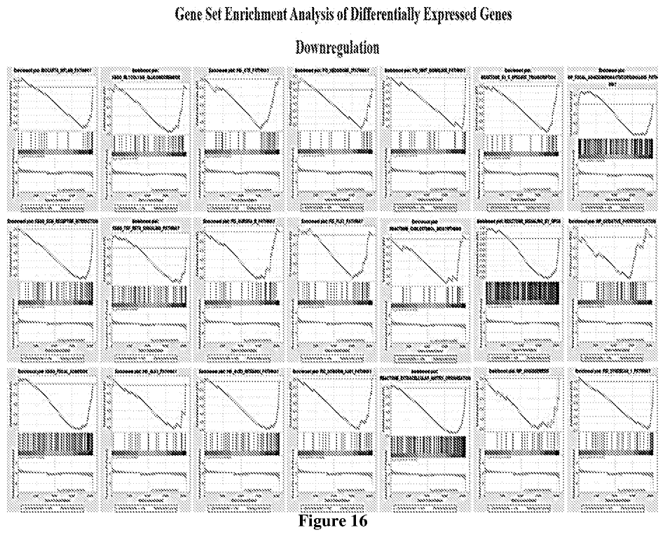

I-8 treatment downregulated many signaling pathways that are critical in the cell death process. Gene Set Enrichment analysis was conducted by comparing the downregulated genes with canonical set of target genes and identified the downregulated pathways. The major downregulated pathways identified upon I-8 treatment are inflammation, glycolysis/gluconeogenesis, Wnt signaling, TGF beta signaling, integrin signaling, Hedgehog pathway, angiogenesis, syndecan1 pathway, FAK mediated PI3K/Akt/mTOR signaling pathway etc. (FIG. 16).

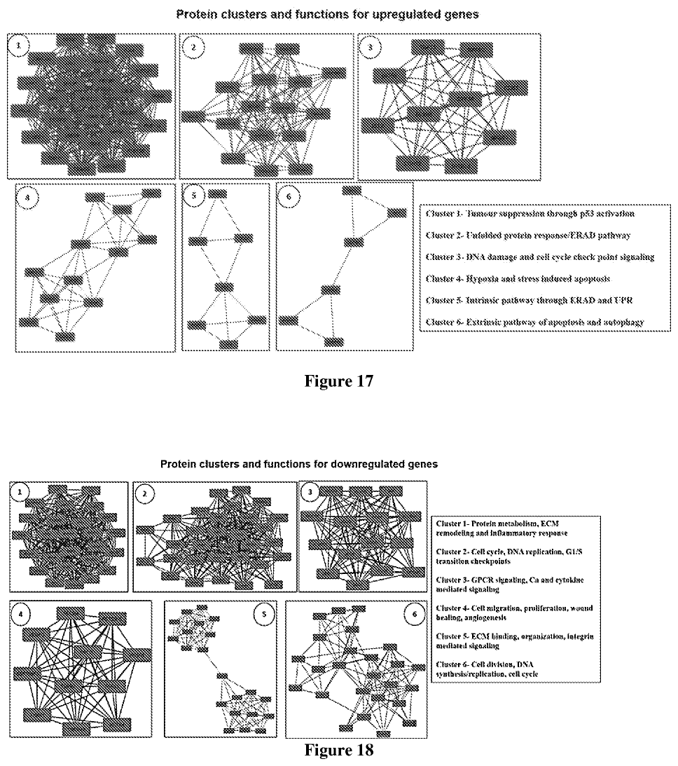

I-8 treatment resulted in the clustering (6 major clusters) of upregulated targeted genes coding proteins which are required tumour suppression and stress induced apoptosis and autophagy (FIG. 17).

I-8 treatment resulted in the clustering (6 major clusters) of downregulated target genes coding proteins which are required for downregulation of integrin mediated signaling and cell cycle arrest (FIG. 18).

I-8 enhances the chemotherapeutic potential of the standard drug paclitaxel.

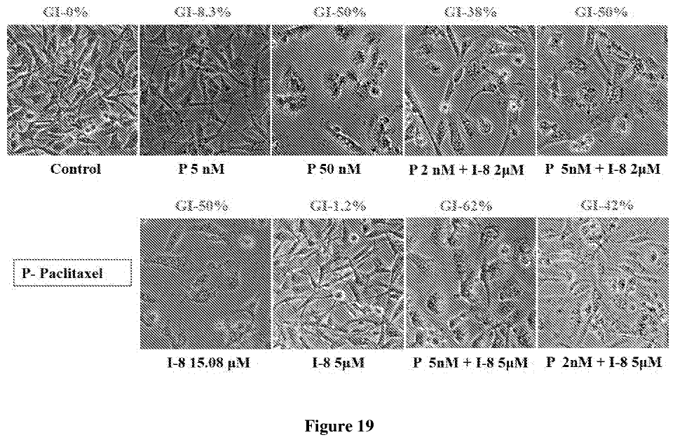

The growth inhibitory studies revealed that, use of 5 μM concentration of I-8 reduced the required concentration of paclitaxel (for 50% growth inhibition) to a 10 times lower concentration than the original concentration (FIG. 19).

Although the subject matter has been described with reference to specific embodiments, this description is not meant to be constructed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The following examples are given by way of illustration of the working of the disclosure in actual practice and therefore should not be construed to limit the scope of the present disclosure.

### Example 1

### N-benzyl-3-(3,4,5-trimethoxyphenyl)-1H-pyrazole-5-carboxamide (I-1)

1 equiv. of benzylamine (26.79 mg, 0.027 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. . . . The solution was stirred at 25° C. for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-benzyl-3-(3,4,5-trimethoxyphenyl)-1H-pyrazole-5-carboxamide was obtained as white solid; 78 mg (84%).

¹H NMR (500 MHz, CDCl₃) δ 7.29-7.28 (m, 3H), 7.26 (s, 1H), 6.96 (s, 1H), 6.82 (s, 2H), 4.60 (d, J=6.0 Hz, 2H), 3.84 (s, 3H), 3.82 (s, 6H). ¹³C NMR (125 MHz, CDCl₃) δ 161.9, 153.6, 138.5, 137.8, 128.6, 127.6, 127.5, 125.1, 102.9, 96.1, 60.8, 55.9, 43.3. HRMS (ESI) (m/z): Calcd for C₂₀H₂₁N₃O₄, (M+H)⁺: 368.16103; Found: 368.16069.

### Example 2

### N-(4-methylbenzyl)-3-(3,4,5-trimethoxyphenyl)-1H-pyrazole-5-carboxamide (I-2)

1 equiv. of 4-methylbenzylamine (30.29 mg, 0.032 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(4-methylbenzyl)-3-(3,4,5-trimethoxyphenyl)-1H-pyrazole-5-carboxamide was obtained as white solid; 92 mg (96%).

¹H NMR (500 MHZ, CDCl₃) δ 7.20 (d, J=8.0 Hz, 2H), 7.09 (d, J=8.0 Hz, 2H), 6.95 (s, 1H), 6.82 (s, 2H), 4.56 (d, J=6.0 Hz, 2H), 3.84 (s, 3H), 3.82 (s, 6H), 2.30 (s, 3H). ¹³C NMR (126 MHZ, CDCl₃) δ 161.9, 153.6, 138.4, 137.1, 134.7, 129.3, 127.6, 102.9, 96.4, 60.8, 55.9, 43.1, 21.1. HRMS (ESI) (m/z): Calcd for C₂₁H₂₃N₃O₄, (M+H)⁺ 382.17668; Found: 382.17592.

### Example 3

### N-(4-methoxybenzyl)-3-(3,4,5-trimethoxyphenyl)-1H-pyrazole-5-carboxamide (I-3)

1 equiv. of 4-methoxybenzylamine (34.29 mg, 0.032 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(4-methoxybenzyl)-3-(3,4,5-trimethoxyphenyl)-1H-pyrazole-5-carboxamide was obtained as white solid; 78 mg (79%).

¹H NMR (500 MHZ, Acetone) δ 12.87 (s, 1H), 7.95 (s, 1H), 7.33 (d, J=7.5 Hz, 2H), 7.15-7.12 (m, 3H), 6.89 (d, J=7.5 Hz, 2H), 4.54 (d, J=5.5 Hz, 2H), 3.90 (s, 6H), 3.77 (s, 6H). ¹³C NMR (126 MHZ, Acetone) δ 158.9, 153.9, 128.9, 113.7, 102.9, 59.7, 55.6, 54.6, 41.8. HRMS (ESI) (m/z): Calcd for C₂₁H₂₃N₃O₅, (M+H)⁺: 398.17160; Found: 398.17049.

### Example 4

### N-(4-chlorobenzyl)-3-(3,4,5-trimethoxyphenyl)-1H-pyrazole-5-carboxamide (I-4)

1 equiv. of 4-chlorobenzylamine (35.40 mg, 0.030 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(4-chlorobenzyl)-3-(3,4,5-trimethoxyphenyl)-1H-pyrazole-5-carboxamide was obtained as white solid; 81 mg (81%).

¹H NMR (500 MHZ, CDCl₃) δ 7.25-7.24 (m, 4H), 6.96 (s, 1H), 6.80 (s, 2H), 4.57 (d, J=6.0 Hz, 2H), 3.85 (s, 3H), 3.83 (s, 6H). ¹³C NMR (126 MHZ, DMSO) δ 161.8, 153.3, 147.6, 143.6, 138.9, 137.6, 131.2, 129.2, 129.1, 128.3, 128.1, 124.3, 102.9, 60.1, 56.1, 55.7, 41.3. HRMS (ESI) (m/z): Calcd for C₂₀H₂₀ClN₃O₄, (M+H)⁺ 402.12206; Found: 402.12255.

### Example 5

### N-(4-fluorobenzyl)-3-(3,4,5-trimethoxyphenyl)-1H-pyrazole-5-carboxamide (I-5)

1 equiv. of 4-flourobenzylamine (31.28 mg, 0.028 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(4-flourobenzyl)-3-(3,4,5-trimethoxyphenyl)-1H-pyrazole-5-carboxamide was obtained as white solid; 72 mg (75%).

¹H NMR (500 MHZ, Acetone) δ 12.70 (s, 1H), 7.96 (s, 1H), 7.29 (dd, J=8.5, 5.5 Hz, 2H), 7.00 (s, 3H), 6.94 (t, J=9.0 Hz, 2H), 4.45 (d, J=6.5 Hz, 2H), 3.75 (s, 6H), 3.61 (s, 3H). 13C NMR (126 MHz, Acetone) δ 162.8, 160.9, 153.9, 129.5, 129.5, 114.9, 114.8, 103.0, 59.7, 55.6, 41.6. HRMS (ESI) (m/z): Calcd for C₂₀H₂₀FN₃O₄, (M+H)⁺ 386.15161; Found: 386.15124.

### Example 6

### N-(2-chlorobenzyl)-3-(3,4,5-trimethoxyphenyl)-1H-pyrazole-5-carboxamide (I-6)

1 equiv. of 2-chlorobenzylamine (35.40 mg, 0.030 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(2-chlorobenzyl)-3-(3,4,5-trimethoxyphenyl)-1H-pyrazole-5-carboxamide was obtained as white solid: 91 mg (91%).

¹H NMR (500 MHZ, Acetone) δ 12.82 (s, 1H), 8.02 (s, 1H), 7.33 (d, J=8.5 Hz, 1H), 7.29-7.27 (m, 1H), 7.19-7.13 (m, 2H), 7.01 (s, 3H), 4.56 (d, J=6.0 Hz, 2H), 3.77 (s, 6H), 3.62 (s, 3H). ¹³C NMR (125 MHZ, Acetone) δ 153.9, 138.6, 136.7, 132.6, 129.2, 128.9, 128.6, 127.1, 103.0, 102.5, 59.8, 55.7, 40.3. HRMS (ESI) (m/z): Calcd for C₂₀H₂₀ClN₃O₄, (M+H)⁺: 402.12206; Found: 402.12154.

### Example 7

### N-(3,4-dichlorobenzyl)-3-(3,4,5-trimethoxyphenyl)-1H-pyrazole-5-carboxamide (I-7)

1 equiv. of 3,4-dichlorobenzylamine (44.01 mg, 0.033 ml, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(3,4-dichlorobenzyl)-3-(3,4,5-trimethoxyphenyl)-1H-pyrazole-5-carboxamide was obtained as white solid; 69 mg (63%).

¹H NMR (500 MHZ, Acetone) δ 13.16 (s, 1H), 8.40 (s, 1H), 7.58 (d, J=2.0 Hz, 1H), 7.49 (d, J=8.5 Hz, 1H), 7.36 (dd, J=8.5, 2.0 Hz, 1H), 7.17 (s, 2H), 7.14 (s, 1H), 4.62 (d, J=6.0 Hz, 2H), 3.90 (s, 6H), 3.76 (s, 3H). ¹³C NMR (125 MHZ, Acetone) δ 153.9, 141.1, 138.7, 131.6, 130.4, 130.1, 129.6, 127.7, 103.1, 102.5, 59.7, 55.7, 41.4. HRMS (ESI) (m/z): Calcd for C₂₀H₁₉C₁₂N₃O₄, (M+H)⁺ 436.08309; Found: 436.08256.

### Example 8

### N-(3,5-bis(trifluoromethyl)benzyl)-3-(3,4,5-trimethoxyphenyl)-1H-pyrazole-5-carboxamide (I-8)

1 equiv. of 3,5-bis(trifluoromethyl)benzylamine (60.78 mg, 0.25 mmol) was added to a mixture of EDC·HCl (57.51 mg, 0.3 mmol), HOBt (40.53 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 6-7 hrs. After the completion of the reaction, the reaction mixture was extracted with DCM. The organic layer was dried over anhydrous Na₂SO₄ and solvent was evaporated in vacuo. The residue on silica gel (100-200) column chromatography using 50% ethyl acetate and hexane as the eluents yielded the product. The compound N-(3,5-bis(trifluoromethyl)benzyl)-3-(3,4,5-trimethoxyphenyl)-1H-pyrazole-5-carboxamide was obtained as white solid; 108 mg (86%).

¹H NMR (500 MHZ, Acetone) δ 12.72 (s, 1H), 8.34 (s, 1H), 7.92 (s, 2H), 7.78 (s, 1H), 6.99 (d, J=5.5 Hz, 3H), 4.68 (d, J=6.0 Hz, 2H), 3.75 (s, 6H), 3.62 (s, 3H). ¹³C NMR (126 MHz, Acetone) δ 153.9, 143.5, 138.6, 131.2, 130.9, 128.3, 124.7, 122.6, 120.7, 120.6, 120.6, 103.1, 102.6, 59.8, 55.7, 41.8. HRMS (ESI) (m/z): Calcd for C₂₂H₁₉F₆N₃O₄, (M+H)⁺ 504.13580; Found: 504.13430.

### Example 9

### N-(phenylsulfonyl)-3-(3,4,5-trimethoxyphenyl)-1H-pyrazole-5-carboxamide (I-9)

1 equiv. of benzenesulfonamide (39.29 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-(phenylsulfonyl)-3-(3,4,5-trimethoxyphenyl)-1H-pyrazole-5-carboxamide was obtained as pale-yellow solid: 37 mg (35%).

¹H NMR (500 MHZ, CDCl₃) δ 11.82 (s, 1H), 9.78 (s, 1H), 8.15 (d, J=8.0 Hz, 2H), 7.65 (t, J=7.5 Hz, 1H), 7.55 (t, J=8.0 Hz, 2H), 6.97 (s, 1H), 6.77 (s, 2H), 3.85 (s, 9H). ¹³C NMR (126 MHZ, MeOD) δ 153.7, 133.4, 128.6, 127.8, 103.4, 102.9, 59.8, 55.4. HRMS (ESI) (m/z): Calcd for C₁₉H₁₉ClN₃O₆S, (M+H)⁺ 418.10728; Found: 418.10637.

### Example 10

### N-((4-methoxyphenyl) sulfonyl)-3-(3,4,5-trimethoxyphenyl)-1H-pyrazole-5-carboxamide (I-10)

1 equiv. of 4-methoxybenzenesulfonamide (46.80 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((4-methoxyphenyl) sulfonyl)-3-(3,4,5-trimethoxyphenyl)-1H-pyrazole-5-carboxamide was obtained as pale yellow solid; 38 mg (34%).

¹H NMR (500 MHZ, DMSO) δ 13.91 (s, 1H), 13.29 (s, 1H), 7.92 (s, 2H), 7.20-7.09 (m, 4H), 5.75 (s, 1H), 3.84 (s, 9H), 3.67 (s, 3H). ¹³C NMR (125 MHZ, DMSO) δ 160.9, 159.6, 153.7, 149.9, 130.3, 118.2, 104.3, 60.5, 56.4, 56.1, 55.4. HRMS (ESI) (m/z): Calcd for C₂₀H₂₁N₃O₇S, (M+H)⁺ 448.11785; Found: 448.11839.

### Example 11

### N-((4-chlorophenyl) sulfonyl)-3-(3,4,5-trimethoxyphenyl)-1H-pyrazole-5-carboxamide (I-11)

1 equiv. of 4-chlorobenzenesulfonamide (47.91 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((4-chlorophenyl) sulfonyl)-3-(3,4,5-trimethoxyphenyl)-1H-pyrazole-5-carboxamide was obtained as pale yellow solid; 46 mg (41%).

¹H NMR (500 MHZ, CDCl₃) δ 9.72 (bs, 1H), 8.10 (d, J=9.0 Hz, 2H), 7.52 (d, J=8.5 Hz, 2H), 7.26 (s, 1H), 6.98 (s, 1H), 6.76 (s, 2H), 3.87 (s, 9H). ¹³C NMR (125 MHZ, DMSO) δ 160.8, 153.2, 129.5, 128.8, 104.0, 102.7, 60.1, 55.9. HRMS (ESI) (m/z): Calcd for C₁₉H₁₈ClN₃O₆S, (M+H)⁺ 452.06831; Found: 452.06874.

### Example 12

### N-((4-bromophenyl) sulfonyl)-3-(3,4,5-trimethoxyphenyl)-1H-pyrazole-5-carboxamide (I-12)

1 equiv. of 4-bromobenzenesulfonamide (59.02 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((4-bromophenyl) sulfonyl)-3-(3,4,5-trimethoxyphenyl)-1H-pyrazole-5-carboxamide was obtained as pale yellow solid; 64 mg (52%).

¹H NMR (500 MHZ, DMSO) δ 13.96 (s, 1H), 13.37 (s, 1H), 7.90-7.83 (m, 4H), 7.24 (s, 1H), 7.10 (s, 2H), 3.83 (s, 6H), 3.67 (s, 3H). ¹³C NMR (125 MHZ, DMSO) δ 162.8, 161.6, 161.0, 153.7, 132.3, 130.1, 104.6, 103.2, 60.6, 56.4. HRMS (ESI) (m/z): Calcd for C₁₉H₁₈BrN₃O₆S, (M+H)⁺ 496.01779; Found: 496.01878.

### Example 13

### N-((4-(trifluoromethyl)phenyl)sulfonyl)-3-(3,4,5-trimethoxyphenyl)-1H-pyrazole-5-carboxamide (I-13)

1 equiv. of 4-(trifluoromethyl)benzenesulfonamide (56.29 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((4-(trifluoromethyl)phenyl)sulfonyl)-3-(3,4,5-trimethoxyphenyl)-1H-pyrazole-5-carboxamide was obtained as pale yellow solid; 59 mg (49%).

¹H NMR (500 MHz, DMSO) δ 13.29 (s, 1H), 8.17 (d, J=7.0 Hz, 2H), 7.98 (s, 2H), 7.20 (s, 1H), 7.10 (s, 2H), 3.83 (s, 6H), 3.67 (s, 3H). ¹³C NMR (126 MHz, DMSO) δ 153.7, 128.9, 126.3, 104.6, 103.1, 60.5, 56.4. HRMS (ESI) (m/z): Calcd for C₂₀H₁₈F₃N₃O₆S, (M+H)⁺ 486.09467; Found: 486.09550.

### Example 14

### N-((3-(trifluoromethyl)phenyl)sulfonyl)-3-(3,4,5-trimethoxyphenyl)-1H-pyrazole-5-carboxamide (I-14)

1 equiv. of 3-(trifluoromethyl)benzenesulfonamide (56.29 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((3-(trifluoromethyl)phenyl)sulfonyl)-3-(3,4,5-trimethoxyphenyl)-1H-pyrazole-5-carboxamide was obtained as white solid; 32 mg (26%).

¹H NMR (500 MHZ, DMSO) δ 8.29-8.26 (m, 2H), 8.06 (s, 1H), 7.87 (s, 1H), 7.24 (s, 1H), 7.11 (s, 2H), 3.83 (s, 6H), 3.67 (s, 3H). ¹³C NMR (126 MHZ, DMSO) δ 153.2, 131.5, 130.3, 124.3, 104.2, 102.7, 60.0, 55.9. HRMS (ESI) (m/z): Calcd for C₂₀H₁₈F₃N₃O₆S, (M+H)⁺ 486.09467; Found: 486.09547.

### Example 15

### N-((2-bromophenyl) sulfonyl)-3-(3,4,5-trimethoxyphenyl)-1H-pyrazole-5-carboxamide (I-15)

1 equiv. of 2-bromobenzenesulfonamide (59.02 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((2-bromophenyl) sulfonyl)-3-(3,4,5-trimethoxyphenyl)-1H-pyrazole-5-carboxamide was obtained as white solid: 58 mg (47%).

¹H NMR (500 MHz, CDCl₃) δ 11.63 (s, 1H), 10.14 (s, 1H), 8.45 (d, J=7.5 Hz, 1H), 7.73 (d, J=7.5 Hz, 1H), 7.58 (t, J=8.0 Hz, 1H), 7.50 (t, J=7.0 Hz, 1H), 6.97 (s, 1H), 6.79 (s, 2H), 3.87 (s, 9H). ¹³C NMR (126 MHz, CDCl₃) δ 159.0, 153.9, 145.6, 145.2, 138.9, 137.4, 135.3, 135.1, 133.6, 127.9, 123.5, 120.2, 104.3, 102.9, 61.0, 56.23. HRMS (ESI) (m/z): Calcd for C₁₉H₁₈BrN₃O₆S, (M+H)⁺ 496.01779; Found: 496.01787.

### Example 16

### N-((5-chlorothiophen-2-yl) sulfonyl)-3-(3,4,5-trimethoxyphenyl)-1H-pyrazole-5-carboxamide (I-16)

1 equiv. of 5-chlorothiophene-2-sulfonamide (49.42 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((5-chlorothiophen-2-yl) sulfonyl)-3-(3,4,5-trimethoxyphenyl)-1H-pyrazole-5-carboxamide was obtained as white solid: 39 mg (34%).

¹H NMR (500 MHZ, DMSO) δ 7.62 (s, 1H), 7.24-7.22 (m, 2H), 7.11 (s, 2H), 3.84 (s, 6H), 3.68 (s, 3H). ¹³C NMR (126 MHZ, DMSO) δ 161.2, 153.2, 137.5, 127.1, 104.1, 102.7, 60.1, 55.9. HRMS (ESI) (m/z): Calcd for C₁₇H₁₆ClN₃O₆S2, (M+H)⁺ 458.02473; Found: 458.02565.

### Example 17

### N-((4,5-dichlorothiophen-2-yl) sulfonyl)-3-(3,4,5-trimethoxyphenyl)-1H-pyrazole-5-carboxamide (I-17)

1 equiv. of 4,5-dichlorothiophene-2-sulfonamide (58.02 mg, 0.25 mmol) was added to a mixture of EDC·HCl (57.51 mg, 0.3 mmol), DMAP (30.54 mg, 0.3 mmol) and 3-(3,4,5-trimethoxyphenyl)-1H-pyrazole-5-carboxylic acid (70 mg, 0.25 mmol), obtained by the hydrolysis of ethyl 3-(3,4,5-trimethoxyphenyl)-1H-pyrazole-5-carboxylate which was prepared by the method described by (A. Kamal, A. B. Shaik, B. B. Rao, I. Khan, G. B. Kumara and N. Jain, Org. Biomol. Chem., 2015, 13, 10162-10178.), in DCM (2 ml) at 25° C. The solution was stirred at room for 16 hrs. After the completion of the reaction, IM HCl (3 ml) was added to the reaction mixture. The reaction mixture was then extracted with DCM. The organic layer was dried over anhydrous MgSO₄ and solvent was evaporated in vacuo. The residue on silica gel (100-200) column chromatography using 70% ethyl acetate and hexane as the eluents yielded the product. The compound N-((4,5-dichlorothiophen-2-yl) sulfonyl)-3-(3,4,5-trimethoxyphenyl)-1H-pyrazole-5-carboxamide was obtained as white solid; 63 mg (64%).

¹H NMR (500 MHZ, DMSO) δ 7.69 (s, 1H), 7.18 (s, 1H), 7.11 (s, 2H), 3.84 (s, 6H), 3.68 (s, 3H). ¹³C NMR (126 MHz, DMSO) δ 162.8, 153.7, 137.8, 130.5, 123.1, 104.5, 103.1, 60.5, 56.4. HRMS (ESI) (m/z): Calcd for C₁₇H₁₅Cl₂N₃NaO₆S₂, (M+H)⁺ 513.96770; Found: 513.96866.

### Cell Culture and Treatment

The cells used for the study, human breast adenocarcinoma (MDA-MB-231) and normal human lung fibroblast cells (WI 30) were procured from NCCS, Pune, India, and ATCC, VA, USA, respectively. Cells were maintained in standard culture conditions in DMEM supplemented with 10% FBS and 100 units/ml penicillin/streptomycin under 5% CO₂ at 37° C. in a humidified incubator. After 2-3 passages, the cells were seeded on cell culture dishes and were used for different experiments.

A 10 mM stock solution of I-8 was prepared in DMSO and diluted with DMEM supplemented with 5% FBS for each experiment so that the DMSO concentration was <0.1% in the treated cells. Paclitaxel at a concentration of 50 nM was used as the positive control.

### Cytotoxicity Studies by MTT Assay

MTT assay was used for testing the cytotoxicity of NIIST-AC-1 in MDA MB 231 cells. 1×10⁴ cells/well were seeded in 96 well culture plates and were treated with different concentrations of NIIST-AC-1 for 24 hrs. The percentage of viable cells were monitored based on the purple colour developed by MTT at 570 nm and from that the concentration of NIIST-AC-1 required for 50% inhibition of the viability was calculated as the GI₅₀ value.

### Morphological Analysis by Phase Contrast and Electron Microscopy

Morphological analysis of the cells treated with NIIST-AC-1 at different concentrations were monitored using phase contrast microscope Nikon EclipseTS100 (Nikon Instruments Inc., Melville, NY, USA, Magnification 20×) and SEM (Zeiss EVO 18 Special Edition) at ×2k and ×5k magnification.

### Nuclear Fragmentation by DAPI Staining

Fragmentation of the nucleus on treatment with NIIST-AC-1 was analyzed using the cell permeable dye which binds to AT rich sequences in the minor groove of DNA. After treatment in 96 well culture plates for 24 hr, the cells were stained with DAPI for 20 min. The morphology of the stained nucleus was observed using a spinning disc fluorescent microscope at an excitation/emission of 360/460 nm (BD Pathway™ 855; Biosciences), and images were taken using the software BD Atto Vision™ version 1.6.

### Phosphatidyl Serine Translocation by Annexin V Staining

The translocation of phosphatidyl serine to the outer leaflet of plasma membrane during apoptosis was studied by Annexin V-Cy3™ Apoptosis Detection Kit. After treatment with different concentrations of NIIST-AC-1 for 24 hrs, the cells were washed and stained with annexin V for 10 min. After the staining solution was removed, the binding buffer was added and the cells observed under a spinning disc fluorescent microscope (BD Biosciences), and images were taken using the software BD Atto Vision version 1.6.

### Assay of Caspase 3 and Caspase 9

The activities of the initiator caspase-9 and downstream executioner caspase-3 were measured using Fluorometric Assay Kit. The caspase activity in the cell lysates of control and treated cells were measured by using the fluorescent substrate DEVD-AFC (for caspase-3 activity) and LEHD-AFC (for caspase-9 activity). Samples were read at 400-nm excitation filter and 505-nm emission filter in a fluorescent microplate reader (BioTek Synergy HT) using Gen5™ ver 1.05.11 software, and the results were expressed as relative units in terms of fluorescence.

### Measurement of Intracellular Reactive Oxygen Species (ROS)

The upregulation of ROS upon treatment with NIIST-AC-1 was analyzed using the fluorescent probe CM-H2DCFDA (Sigma). After treatment, the cells were incubated DMEM containing CM-H2DCFDA at a final concentration of 5 μM at 37° C. for 30 min. The trypsinised cells were then suspended in PBA and analyzed using FACS (BD FACSAria II (BD Biosciences) at fluorescein isothiocyanate (FITC) range (excitation 495 nm, emission 529 nm bandpass filter). The mean fluorescence intensities of different groups were analyzed by BD FACSDiva ver 6.1.3 software and corrected for autofluorescence from unlabeled cells.

### Measurement of Mitochondrial Membrane Potential

JC-1 Mitochondrial Membrane Potential Assay Kit was used for the Fluorescent microscopic studies on loss of mitochondrial membrane potential in the treated cells. The cells were then treated with various concentrations of NIIST-AC-1 for 24 h. After removing the spent medium, 100 μl of freshly prepared JC-1 stain was added and samples were then incubated for 20 min in a CO2 incubator at 37° C. Immediately after the incubation, cells were observed under a spinning disc fluorescent microscope (BD Pathway 855), and images were taken using the software BD Atto Vision version 1.6.

### Protein Array Experiment for the Expression of Apoptotic Proteins

The expression of various proteins in the extrinsic and intrinsic pathway of apoptosis on treatment with NIIST-AC-1 was analyzed by using antibody pair-based assay, where the 43 targets were captured on a membrane. Lysates of control and treated cells were spotted to the membrane as protein equivalents. Paired biotinylated detector antibodies and streptavidin HRP were used for development using chemiluminescent method. Comparison of the membrane were done using densitometry software.

### Anoikis Assay

The effect of NIIST-AC-1 on anoikis resistance was analyzed using Anoikis Assay Kit by following the manufacturer's protocol. The cells were seeded in ultralow attachment plate 9anoikis chamber) after pretreating with different concentrations of NIIST-AC-1. After 48 hrs, the cells were observed under the phase-contrast microscope (Nikon Eclipse TS100) for observing the attachment and images were captured using NIS-Elements 3.21.00 imaging software. The viability of the cells was also measured by MTT assay.

### Cell Cycle Analysis

The distribution of cells in different phases of cell cycle was determined by flow cytometry after staining with propidium iodide. After treatment, the cells were harvested, ethanol fixed and stained with propidium iodide at 25° C. for 30 minutes. The cellular DNA content was measured with BD FACS Fortessa X-20 SORP according to detected signals in FL2 channel (excitation, 493 nm; emission, 636 nm) and data were analyzed with FloJo software, version 9. Approximately 10,000 cells were counted for each analysis, and the distribution of cells in each phase of the cell cycle was saved as histograms. Western blot analysis was done to study the expression profiles of various proteins involved in the cell cycle machinery.

### HDAC Inhibition Assay

The histone deacetylase inhibitory activity was measured in the cell lysates using the colorimetric assay kit where we have used the substrate Ac-Lys (Ac)-pNA and trichostatin A as the positive control. After the reaction, the absorbance was measured at 400 nm and the percentage inhibition was calculated by comparing with the deacetylated standard Ac-Lys-pNA.

### Antimigration Potential Measurement

Wound healing assay was performed to understand the antimigration effects of NIIST-AC-1. The confluent monolayers of cells after making the scratch were treated with the test compounds at different concentrations for 24 hr. After the time interval, the wound closure by the migration of the cells was observed under the phase-contrast microscope (Nikon Eclipse TS100), and images were captured using NIS-Elements 3.21.00 imaging software at different time points (0, 24 h) at 4× magnification. The wound area was quantified using ImageJ 1.52p software.

### Colony Formation Assay

The pretreated cells with different concentrations of NIIST-AC-1 for 48 hr were plated in six well plates and allowed to grow and colonize for 14 days. The colonies were stained with crystal violet for 2 hr after fixing with acetic acid and methanol. The visible colonies were photographed and the colon area was quantified using ImageJ 1.52p software.

### Gelatin Zymography for MMP Activity Measurement

The activity of MMPs in the spent medium from the cell culture was allowed to react with the substrate gelatin impregnated in the SDS-PAGE gel. After electrophoresis, the gel was washed with Triton solution and incubated with activation buffer 50 mM Tris containing 0.2 M NaCl and 5 mM CaCl2) at 37° C. for 18-20 h for reaction to occur. The gel was stained with Coomassie Brilliant Blue solution and after destaining, gelatin cleaved zones by MMPs were observed as white clear zones which were imaged using ChemiDoc™ MP System with IMAGE LAB™ Software (Bio-Rad).

### Glucose Uptake Study

The effect of NIIST-AC-1 on the uptake of glucose was analyzed by flow cytometry using 2-NBDG. After incubation of the cells with different concentrations of NIIST-AC-1, the cell culture medium was replaced with medium containing fluorescent 2-NBDG and incubated for 30 minutes. Then the samples were analyzed using BD FACSAria II (BD Biosciences) at FITC range (excitation, 465 nm; emission 540-nm bandpass filter). The mean fluorescence intensities of different groups were analyzed by BD FACSDiva ver 6.1.3 software and corrected for autofluorescence from unlabeled cells.

### EGFR [Epithelial Growth Factor Receptor] Expression Studies

The expression of EGFR after treatment with NIIST-AC-1 was evaluated using indirect immunofluorescence. The cells after treatment were fixed with paraformaldehyde and incubated with primary antibody overnight. After washing the primary antibody, the cells were treated with alexa fluor conjugated secondary antibody for visualization. The cells were counterstained with DAPI for nuclear staining and were observed under a fluorescent microscope (IX83 inverted microscope; Olympus Life Science, cell Sens Dimension ver 3.1 software).

### RNA Sequencing for Whole Transcriptome Analysis

Toral RNA was isolated from the cells using RNeasy mini kit. 15 μg of cellular RNA was quality assessed (Bioanalyzer from Agilent Technologies) and used for mRNA library preparation. The mRNAs were fragmented, and the first strand of cDNA was synthesized from the cleaved RNA using random primers followed by second strand cDNA synthesis. The purified cDNA templates were enriched by PCR amplification to generate cDNA libraries. The developed libraries were presented to RNA sequencing facility and two rapid single-read 50 illumina HiSeq sequencing runs were performed. Raw reads from separate lanes of the same sample were merged for mapping. RNA sequencing was performed at Eurofins Genomics India Pvt. Ltd., Bengaluru 560048, Karnataka, India.

### Identification of Differentially Expressed Genes (DEGs)

The output raw FASTQ files were analyzed using various tools of Galaxy platform to extract the differentially expressed genes as follows. The quality of the reads was analyzed using FastQC (Galaxy Version 0.72+galaxy1). The quality check reports were aggregated using MultiQC (Galaxy Version 1.9+galaxy1). Cutadapt (Galaxy Version 1.16.6) was used for adapter removal and filtering using threshold of minimum read length 20 and minimum quality (phred score) 20. Again, the quality was checked, and results were aggregated. The cleaned reads were mapped to a reference genome (Homo_sapiens.GRCh38.dna_sm.primary_assembly.fa.gz) and annotation file (Homo_sapiens.GRCh38.104.gtf.gz) using HISAT2 (Galaxy Version 2.1.0+galaxy7), a splice-aware alignment program. The output bam files are processed using StringTie (Galaxy Version 2.1.1) to assemble and quantitate RNA-Seq alignments into potential transcript. Later the Deseq2 (Galaxy Version 2.11.40.6+galaxy1) was used to determine normalized differentially expressed genes from the Stringtie output count tables. A total of 26278 differentially expressed gene were obtained in which 12768 were upregulated and 13610 were downregulated. These differentially expressed genes were filtered using Filter (Galaxy Version 1.1.1) to extract significant ones by adding a filter of q-value<=0.05 and log 2 fold change >1. The resulting 1337 upregulated and 1642 downregulated genes were used for further downstream analysis.

### Functional and Pathway Enrichment Analysis

To further understand the biological functions of upregulated and downregulated DEGs, enrichment analysis was performed using the online tool ToppFun from Toppgene suite. The functional annotation (biological process, molecular function and pathways) was considered significant at False discovery rate (FDR B&H)<0.05. GSEA Preranked tool of standalone R program GSEA 4.1.0 was used to perform Gene Set Enrichment Analysis (GSEA) on ranked (log fold change based) list of total DEGs against CP: Canonical pathways (a pre-defined collection of 2922 gene sets) of Molecular Signatures Database (MSigDB). The enriched pathways with FDR qvalue less than 0.25 were considered significant.

### Protein-Protein Interaction Network and Cluster Identification

Proteins and their interactions form a protein-protein interaction network, where the proteins are the nodes, and the interactions are the edges. To visualize the functional interactions of proteins encoded by the identified DEGs, a PPI network was constructed using Search Tool for the Retrieval of Interacting Genes (STRING; version 11.0) with high confidence score of 0.7. This network was then visualized using Cytoscape (version 3.7.1) software and clustered using MCODE plugin (v1.6.1) with default parameters (Degree cut-off, 2; node score cut-off, 0.2; k-core, 2 and max depth, 100) to identify the functional modules. Functional enrichment analysis for these clusters were conducted using the online tool ToppFun from Toppgene suite, and key functions were identified.

### Advantages of the Invention

Breast cancer mortality continues in 2021 also as a major public health problem. Early diagnosis and screening are two important strategies that can improve breast cancer outcomes and survival. Different types of treatment strategies are employed for breast cancer patients such as radiation, surgery, chemotherapy, targeted therapy etc. Triple-negative breast cancer is the most destructive form which lacks progesterone receptor, human epidermal growth factor receptor 2 and estrogen receptor expression. This type is most likely to spread beyond the breast and more likely to recur after treatment. It is more detected in young women, less than 50 years and mostly with an inherited BRCA1 mutation. This is usually treated with a combination strategy with radiation, surgery and chemotherapy. Doxorubicin, cyclophosphamide and paclitaxel are the common chemo drugs for TNBC. Addition of taxanes to anthracycline drugs improved the recurrence risk and mortality when compared with other cytotoxic drugs. Poly ADP-ribose polymerase (PARP) is the enzyme that fixes the DNA damage in both healthy and cancer cells. PARP inhibitors such as Olaparib and talazoparib have been approved to treat advanced TNBC with BRCA1 or BRCA2 mutation. A combination of immune check point (PDL1) inhibitor and chemo drug abraxane (Tecentriq) is also approved to treat locally advanced metastatic triple negative PDL-1 positive breast cancer. In a nut shell, cytotoxic chemotherapy is the backbone of TNBC treatment. But all these drugs are not specific and have lot of side effects. Immunosuppression associated with chemotherapy also leads to several other complications. The inclusion of taxanes creates neurotoxicity and long-term use of anthracyclines cause cardiotoxicity. Hence the identification of small molecules with less toxicity and their use alone or in combination with already known chemo drugs are promising in the future.

The identification and validation of targeted therapy for TNBC patients most urgent in breast cancer therapeutics. A small molecule based anticancer agents with no toxicity against normal cells and which can inhibit the cancerous cells in multiple pathways is need of the hour.

We claim:

1. A pyrazole amide-based compound of formula I 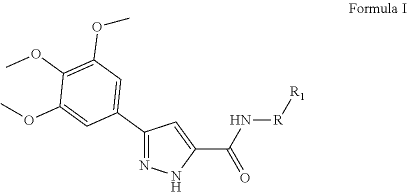
wherein,
when R=—CH₂, then R₁ is selected from the group consisting of benzene, 4-methyl benzene, 4-methoxy benzene, 4-chloro benzene, 4-fluoro benzene, 2-chloro benzene, 3,4-dichloro benzene, and 3,5-trifluoromethyl benzene; and
when R=—SO₂, then R₁ is selected from the group consisting of benzene, 4-methoxy benzene, 4-chloro benzene, 4-bromo benzene, 4-trifluoromethyl benzene, 3-trifluoromethyl benzene, 2-bromo benzene, 2-chlorothiophen or 2,3-dichlorothiophen.

2. The compound as claimed in claim 1, wherein said compound is useful as anticancer agent in triple negative breast cancer (TNBC) cells.

3. The compound as claimed in claim 1, wherein the compound of formula I is selected from the group consisting of:

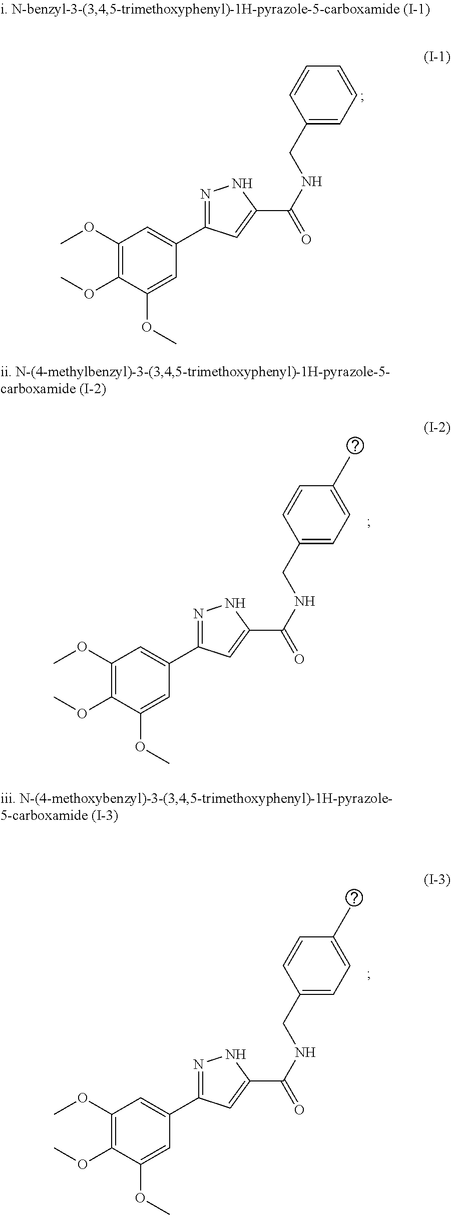

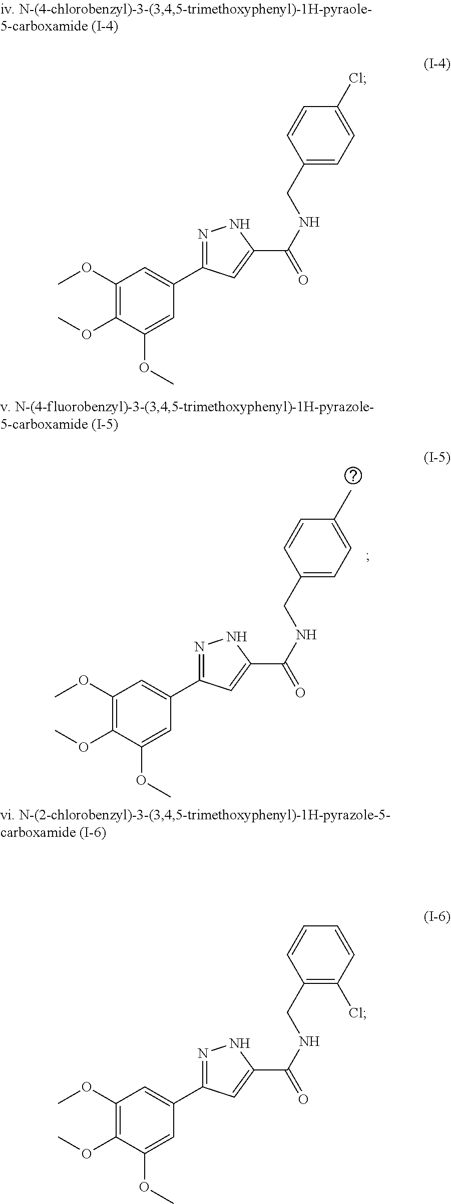

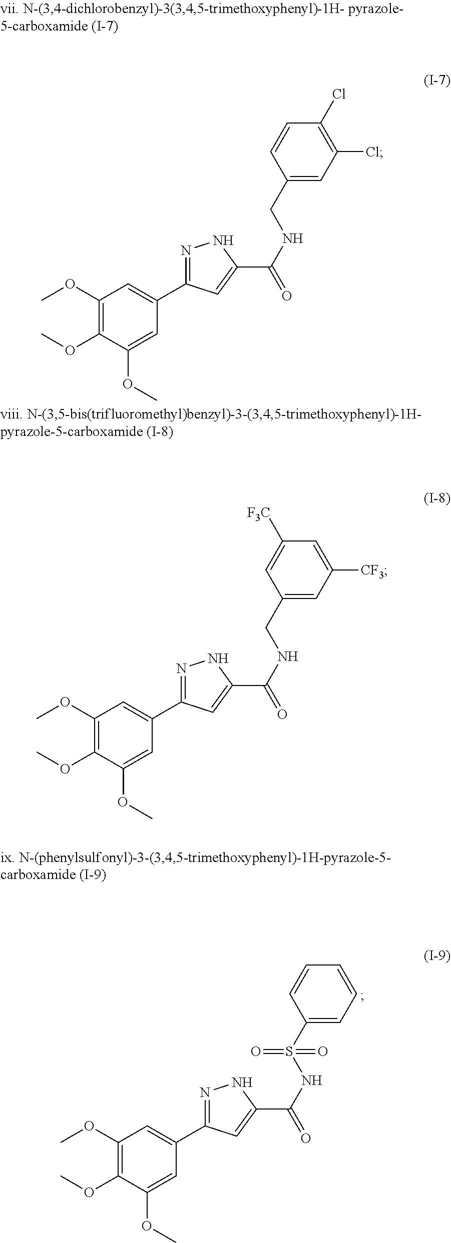 [SMILES: COc1cc(-c2cc(C(=O)NCc3ccc(Cl)c(Cl)c3)[nH]n2)cc(OC)c1OC ; COc1cc(-c2cc(C(=O)NCc3cc(C(F)(F)F)cc(C(F)(F)F)c3)[nH]n2)cc(OC)c1OC ; COc1cc(-c2cc(C(=O)NS(=O)(=O)c3ccccc3)[nH]n2)cc(OC)c1OC]

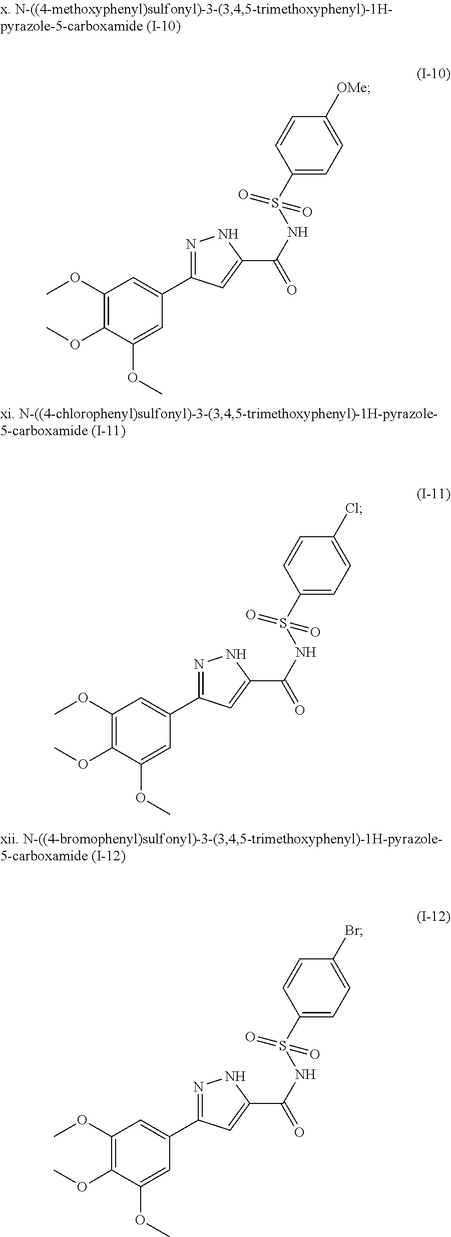 [SMILES: COc1ccc(S(=O)(=O)NC(=O)c2cc(-c3cc(OC)c(OC)c(OC)c3)n[nH]2)cc1 ; COc1cc(-c2cc(C(=O)NS(=O)(=O)c3ccc(Cl)cc3)[nH]n2)cc(OC)c1OC ; COc1cc(-c2cc(C(=O)NS(=O)(=O)c3ccc(Br)cc3)[nH]n2)cc(OC)c1OC]

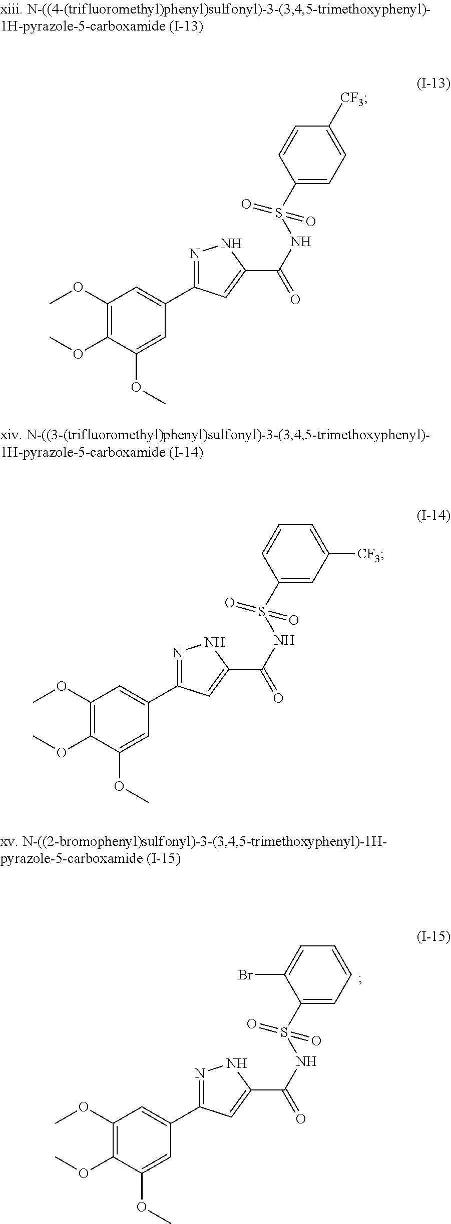 [SMILES: COc1cc(-c2cc(C(=O)NS(=O)(=O)c3ccc(C(F)(F)F)cc3)[nH]n2)cc(OC)c1OC ; COc1cc(-c2cc(C(=O)NS(=O)(=O)c3cccc(C(F)(F)F)c3)[nH]n2)cc(OC)c1OC ; COc1cc(-c2cc(C(=O)NS(=O)(=O)c3ccccc3Br)[nH]n2)cc(OC)c1OC]

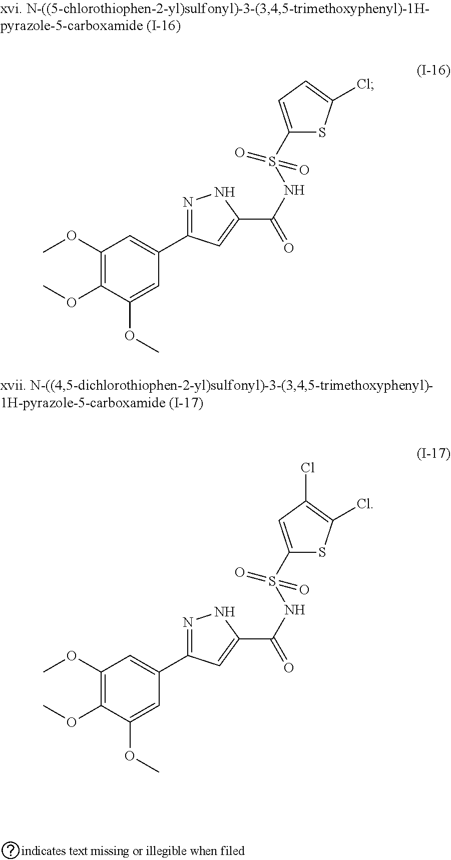 [SMILES: COc1cc(-c2cc(C(=O)NS(=O)(=O)c3ccc(Cl)s3)[nH]n2)cc(OC)c1OC ; COc1cc(-c2cc(C(=O)NS(=O)(=O)c3cc(Cl)c(Cl)s3)[nH]n2)cc(OC)c1OC]

4. A process for the preparation of compound of formula I comprising the steps of:
i. condensing compound of formula 1 with diethyl oxalate to get ethyl 2,4-dioxo-4-(3,4,5-trimethoxyphenyl) butanoate of formula 2; 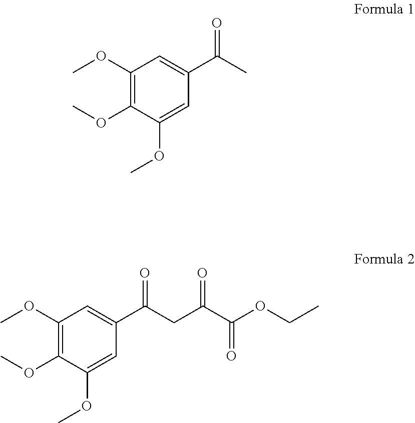 [SMILES: COc1cc(C(C)=O)cc(OC)c1OC ; CCOC(=O)C(=O)CC(=O)c1cc(OC)c(OC)c(OC)c1]
ii. cyclocondensating compound 2 as obtained in step (i) with hydrazine hydrate hydrochloride to form pyrazole ester compound of formula 3; 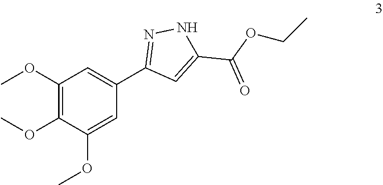 [SMILES: CCOC(=O)c1cc(-c2cc(OC)c(OC)c(OC)c2)n[nH]1]
iii. base mediated hydrolysis of compound of formula 3 as obtained in step (ii) to obtain pyrazole acid compound of formula 4 followed by amide coupling with amine of formula 5 [R1-R—NH₂] to obtain compound of formula I. 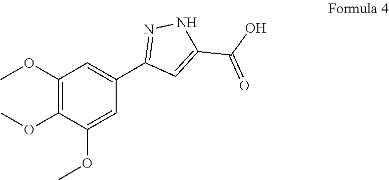
wherein R and R₁ are same as defined in claim 1.
